(19) United States
(12) Patent Application Publication
(10) Pub. No.: US 2011/0087130 A1
(43) Pub. Date: Apr. 14, 2011

(54) METHOD AND SYSTEM FOR DIAGNOSING AND TREATING AUDITORY PROCESSING DISORDERS

(75) Inventors: Randy Cheema, St. John's (CA); Sherry Cheema, St. John's (CA)

(73) Assignee: Systems N' Solutions Ltd., St. John's (CA)

(21) Appl. No.: 12/576,073

(22) Filed: Oct. 8, 2009

(51) Int. Cl. A61B 5/00

(52) U.S. Cl. 600/559

(57) ABSTRACT

A method for diagnosing and treating an auditory processing disorder. The diagnosis phase includes presenting, simultaneously at least one different audible sound in a user's left ear and right ear. Each sound is different for each ear. A determination is made if the user can correctly recall the sound, whereby incorrect recollection indicates a disorder. The treatment phase includes repetitively presenting over time, the patient/user with different audio signals to each ear with the signals being presented simultaneously. An interface is provided for the patient/user to attempt to reproduce said audio signals presented. The patient's/user's ability to accurately reproduce the correct signals over a period of time is monitored with increasing complexity in the exercises.

### FIELD OF THE INVENTION

The present invention relates to a treatment and diagnosis protocol for patients suffering from auditory processing disorders and more particularly, the present invention is directed to the diagnosis and therapeutic process for guiding a patient through a series of simple auditory tests that stimulate the brain to identify and understand the physical characteristics of sound such as pitch, loudness, gaps and duration.

### DESCRIPTION OF THE PRIOR ART

As the name implies, auditory processing disorders relate to the manner in which auditory information is eventually processed within the brain. The disorder does not involve any hearing impairment, but rather an impairment between the brain and the incoming information and how the brain processes such information. Generally speaking, patients with such disorders cannot recognize even subtle differences between the sounds in words, despite the fact that the volume of the sound is adequate to hear.

Listening skills are a very important part of a child's education. In fact, of all the communication skills (reading, viewing, writing, speaking and listening), studies consistently show that listening skills are used most often. Barker et al. in 1980, found that 53% of the time the average person used listening as a communication skill. When a child does not have the skills to listen effectively, he or she cannot process spoken language effectively. This has a dramatic negative impact on their ability to learn under normal classroom conditions.

Even though most of us spend the majority of our day listening, it receives the least instruction in school. Listening training is not required at most universities. Students who are required to take a basic communication course spend less than 7 percent of class and text time on listening.

Children who possess normal listening skills but are limited in their ability to process certain sounds may face frustrating difficulties in one or more of the following situations:
Hearing in noisy situations
Following long conversations
Learning a foreign language or challenging vocabulary words
Remembering spoken information (auditory memory deficits)
Taking notes
Maintaining focus on an activity. Distractions can be experienced if other sounds are present
Following multi-step directions
Directing, sustaining, or dividing attention
Processing nonverbal information (music appreciation)

In order to make sense of the sounds we hear (acoustic signals), the ear converts them to neural signals which are then transmitted along nerve pathways to various parts of the brain (auditory cortex and temporal lobe).

The brain receives these neural signals and identifies the physical characteristics of the original sound. These include frequency, pitch, loudness and duration. This complex processing and interpretation of acoustic signals by the brain is referred to as auditory processing. As with any complex brain process, children can have a broad range of auditory processing abilities and disabilities.

In the auditory processing model, sounds (auditory signals) enter the ear and are converted to neural signals at the cochlea. These neural signals are transmitted along a neural pathway to the auditory cortex on the opposite side of the brain. The left and right auditory cortices are the regions of the brain that process sounds including the spoken word. In childhood, people process signals from both ears, but one side (left or right) is dominant. Normally this dominance lessens as they grow. However, some children with learning disabilities cannot process information using both ears so their auditory processing does not develop normally.

Children as young as two or three years can exhibit symptoms that are often linked to APD. These symptoms include delayed or disordered speech and delayed language skills. Early intervention is critical to proactively foster the child's auditory processing skills and encourage normal language development.

Generally speaking, in order to diagnose a disorder, an audiologic evaluation must be conducted. Such examinations involve tests that can determine the softest sounds and words a person can hear and other tests to see how well recognize sounds in words and sentences are recognized. It is also a common diagnostic technique to have a patient/user listen to different numbers or words in the right and the left ear simultaneously.

Further tasks involve providing two sentences with one being played louder than the other and simultaneously.

Having the diagnostic techniques in place, a number of adaptive training techniques and other listening skill enhancement protocols have been developed. An example of previous techniques includes that which is taught in United States Patent Application Publication No. US2002/0034717, published Mar. 21, 2002, Jenkins et al. This document teaches a method for adaptive training of short term memory and auditory/visual discrimination within a computer game. In this reference, there is disclosed a training method for training auditory and graphical discriminations of humans, and short term memory. This is provided within an animated game environment. The method involves the use of tiles wherein there are a number of stimulus sets, with each set having similar sounding phonemes associated with graphemes. At the onset of a trial, a grid of tiles is presented to a patient. The patient selects the tiles one at a time and as the tiles are selected and associated phoneme is presented to the patient. Subject clears the tiles by pairing them with identical tiles. Upon all of the tiles in a trial being cleared the subject is promoted or demoted in skill level depending on the success.

In U.S. Pat. No. 6,585,517, issued to Wasowicz, Jul. 1, 2003 there is taught a phonological awareness, phonological processing, and reading skill training system and method. In the method for training a user discriminate sound, the method involves presenting a first sound and a second sound to the user with each having a particular set of acoustic components that distinguish the two sounds. The method then involves the receipt of response from the user discriminating the first and second sound. The acoustic component of the first sound is then altered by a prescribed amount in order to make the sound more similar to the second. The method then involves the presentation of the first and second sounds to the user after each alteration of the acoustic components of the first sound where the first sound becomes more difficult to distinguish from the second sound.

### SUMMARY OF THE INVENTION

One object of one embodiment of the present invention is to provide an improved protocol and system for not only diagnosing but improving the listening skills of a patient suffering from auditory processing disorder.

Generally speaking, the system employs listening activities to improve auditory processing and enhanced listening skills. It has been found particularly effective to build reading and language skills in children.

As an example, the protocol can be divided into several distinct modules such as listening skill assessment for purposes of diagnosing the existence of the auditory processing disorder, auditory discrimination training, dichotic digits training, dichotic words training, sound depth detection training, frequency pattern training and ribbon pattern training.

In connection with another object of one embodiment, there is provided a method for diagnosing and treating an auditory processing disorder, comprising:
a diagnosis step including:
presenting, simultaneously at least one different audible sound in a user's left ear and right ear, the sound being different for each said ear;
determining if the user can correctly recall the sound, whereby incorrect recollection indicates a disorder;
a treatment step including:
repetitively presenting over time, the user with different audio signals to each ear, the signals being presented simultaneously;
providing an interface for the user to attempt to reproduce the audio signals presented; and
monitoring the user's ability to accurately reproduce the correct signals over a period of time.

Once the dichotic testing results indicate the presence of a problem, treatment can be instituted by presenting a single different sound to each ear simultaneously for a severely affected child or in pairs for a child less severely afflicted.

As part of the learning process, the child will be able to visually determine his/her progress with an indication of correct responses. This obviously has the benefit of reinforcing the treatment and has a positive impact on the child's self esteem.

Further, as the child progresses through the process, an indication to this effect will be available in the form of a level indicator. This, as an example, may comprise a number indication on the display device and may be combined with an audible indication.

Another object of one embodiment of the present invention is to provide A method for diagnosing and treating auditory processing disorder, comprising:
a diagnosis step and a treatment step, the diagnosis step including:
determining which, if any ear is dominant for hearing in a user by presenting, simultaneously at least on different audible sound in a user's left ear and right ear, the sound being different for each the ear; and
determining if the user can correctly recall the sound in each ear, whereby incorrect recollection indicates a disorder indicating a dominant hearing ear;
a treatment step including;
repetitively presenting over time the user with different audio signals to each ear, the signals being presented simultaneously, presentation being effected in ascending levels with increasingly complex sounds;
varying the volume of repeated signals; providing an interface for the user to attempt to reproduce the audio signals presented; monitoring the user's ability to accurately reproduce the correct signals in each level of the levels, whereby ear dominancy in the user is substantially eliminated.

In order to enhance the effectiveness of the treatment process, the testing may be conducted with background noise simultaneously. This adds another degree of complexity, but has the beneficial consequence of approximating a typical environment where noise is inherent.

Having thus generally described the invention, reference will now be made to the accompanying drawings, illustrating preferred embodiments.

### BRIEF DESCRIPTION OF THE DRAWINGS

FIG. 1 is a schematic illustration of the overall system according to one embodiment;

FIG. 2 is a representative screening shot of the dichotic numbers training module;

FIG. 3 is a screen shot representative of the dichotic words training module;

FIG. 4 is a screen shot representative of the gap detection—temporal skills module;

FIG. 5 is a screen shot representative of the frequency pattern training—temporal skills module; and

FIG. 6 is a screen shot representative of the rhythm pattern training—temporal skills module.

Similar numerals used in the drawings denote similar elements.

### DETAILED DESCRIPTION OF THE INVENTION

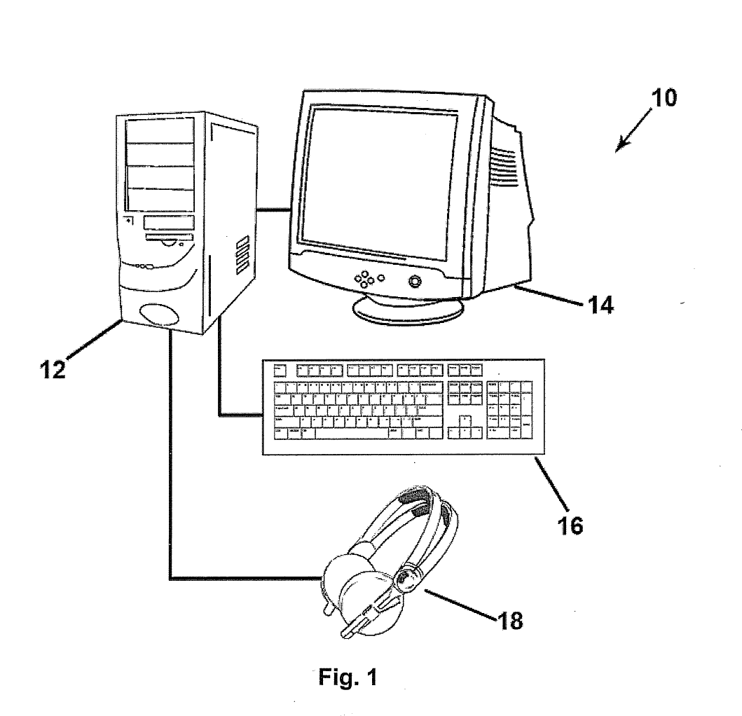

Referring now to FIG. 1, shown is the schematic illustration of the overall system globally denoted by numeral 10. The system includes a CPU 12 which is linked to a monitor 14 for displaying images. The monitor 14 may be of the type that facilitates touch instructions (multi-touch technology or wireless pad) or the system may include a peripheral object such as a keyboard 16. An audio source 18 is linked to the CPU 12 which audio source 18 may comprise speakers of my ear buds, head sets or any other suitable device to convey audible material.

As generically mentioned herein previous, the protocol involves a number of distinct modules, the first of which is a dichotic listening skills assessment module. A use of this module is for assessing a child's dichotic listening skills and ability to process auditory signals. The module is also designed to determine if a dichotic listening problem exists and whether the child/patient's left ear or right ear is dominant.

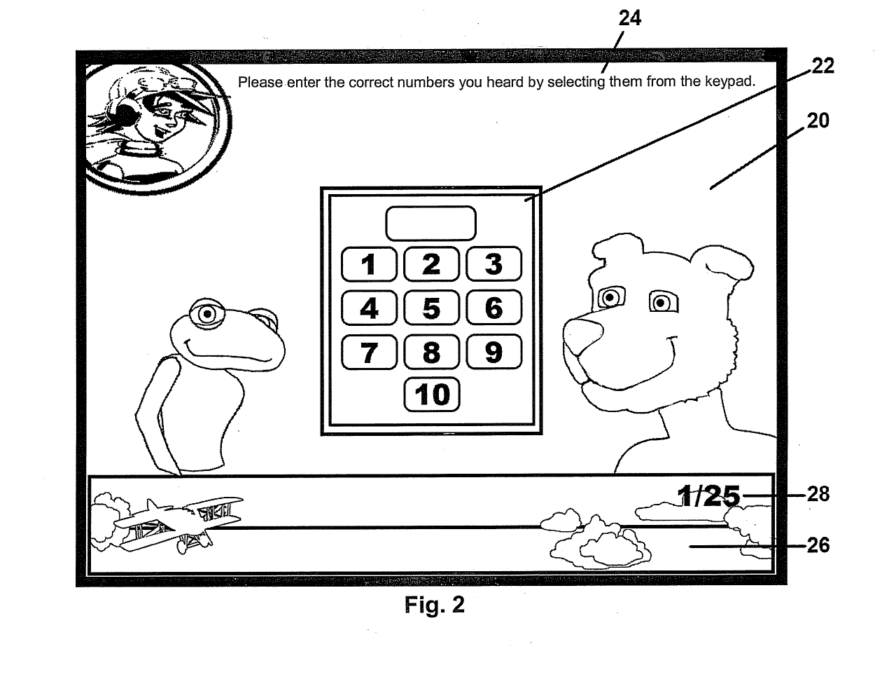

Referring now to FIG. 2, shown is a screen shot for the dichotic numbers training module generally is represented by numeral 20. The screen typically includes a keypad 22 with the numerals 1 through 10 thereon as well as an instruction line 24 which, in this example, indicates the instructions “please enter the correct numbers you heard by selecting them from the keypad.” A progress bar 26 is associated with the bottom portion of the screen and is useful to further endorse the training by depicting the child's progress as it successfully answers questions.

The progress bar will include a number of questions answered correctly versus the number of questions asked

The minimum age for this test is seven years of age with a duration period of 10 minutes for all testing. In the method, different pairs of numbers from one through ten are presented in the child's left and right ears. The child is then asked to input all four numbers.

If the child is unsuccessful in accurately stating the four number, then the dichotic listening problem is evident and the protocol described herein is indicated.

Once diagnosed through the therapeutic aspect of the protocol, then the patient or child is exposed to an auditory discrimination training module. Auditory discrimination is the ability to hear the difference in varying sounds used in speech and then distinguishing between these sounds. Children with auditory discrimination difficulties do not always hear sounds in the correct order or as individual units. For example, if they are asked to spell the word “plan” they may write the word “pan” because they heard the letter grouping “pl” as “p” and did not hear the “l”. This obviously impacts spelling and reading skills. It has been found that children who do not discriminate effectively often have difficulty listening because they have difficulty hearing the words correctly. This condition has an adverse effect on their comprehension and understanding while learning. Unfortunately, these children are sometimes misdiagnosed with attention deficit disorder (“ADD”) or attention deficit hyperactivity disorder (“ADHD”) because they learn to stop paying attention due to the lack of understanding of what is being said. By making use of the discrimination training module children are able to discern one sound from another while reading, spelling and writing. The child also benefits from improved direction following and improved reading skills by enable children to hear the differences between low and quiet sounds, high and low sounds, and long and short sounds. This training also includes a different level of background noise.

If a child has not developed the ability to process the auditory signals from both ears, then he or she is losing up to half of the information available for learning and social interaction. One way to access and enhance the child's auditory processing ability is through dichotic testing and training involves the simultaneous delivery of sounds to one ear that are different from sounds presented to the other ear. The sounds delivered to each ear contain different levels and combinations of frequency, volume and pitch.

With this module, the child's ability to process different auditory signals presented to each ear simultaneously is developed, the child's listening/processing skills in the weaker ear are increased and the child's listening skills are eventually brought to a balanced state. In this module, the different pairs of numbers from one through ten are presented to each ear. The child is then asked to repeat all four numbers. Based on ear dominance the volume is lowered in the stronger ear and is increased in the weaker ear. Each level increases in difficulty by increasing the volume in the child's dominant ear while the volume in the weaker ear is incrementally decreased forcing the weaker ear to work harder until a balanced state is achieved. There are 36 levels in this module with 10 sound tests per level. As the child improves in identification the levels are advanced. There are two levels of dichotic numbers training. Level One is for children more severely challenged auditorially, such as children with autism. Only one number is presented to the right ear and a different number is presented to simultaneously to the left ear as the child's listening skills improve he or she advances to the next level.

Level Two would be a typical starting point for most children. A pair of numbers is presented to the right ear and a separate pair of different numbers is presented simultaneously to the left ear. This training also includes a different level of background noise.

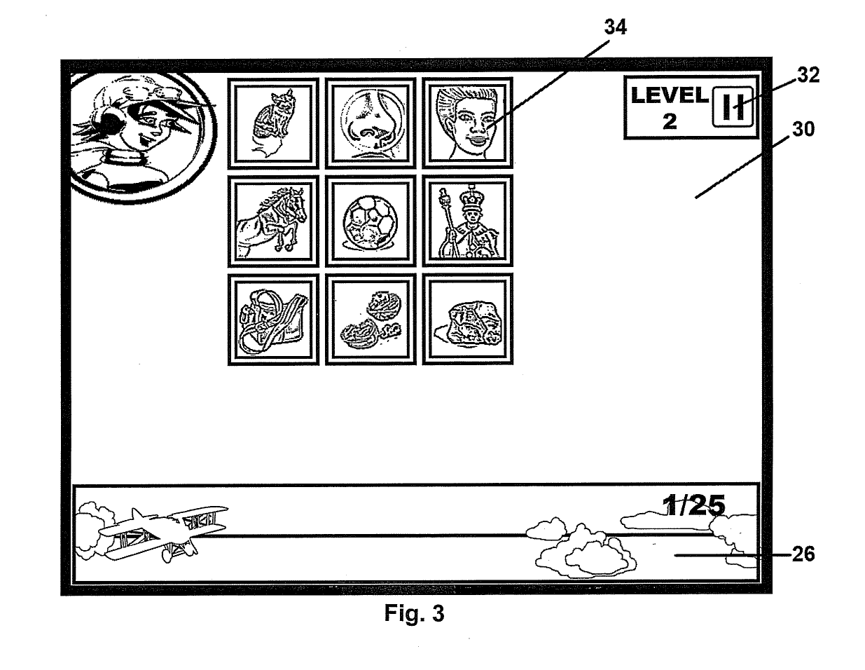

In a further module, there is a dichotic words training module the goal of which is to assess and enhance the listener's ability to process different one syllable words presented to each ear at the same time and to slowly bring the child's hearing to a balanced state. Referring now to FIG. 3, shown is the dichotic words training module screen shot, globally denoted by numeral 30. In this screen shot module the screen provides for a level indicator 32 indicative of the level the child is currently operating in, together with an array of pictures 34 located on the screen. Similar to FIG. 2, progress bar arrangement 26 is provided and functions in the same manner as that denoted with respect to FIG. 2. The module involves an interactive pre-test followed by five test levels with ten dichotic tests per level. The protocol involves a pre-test to determine ear dominance and the child begins at the level appropriate to his or her listening ability. Different pairs of one syllable words are presented to each ear and the listener is asked to select the corresponding pictures from a template of images presented on the monitor screen (not shown). Each level increases in difficulty by decreasing the volume and the child's stronger ear while increasing the volume in the child's weaker ear from level to level. The number of images from which to choose the correct image also increases. After all dichotic word levels are completed, the child does a post-module performance test and the results are compared with the pre-test results to measure the child's improvement in listening and auditory processing skills. As with the other modules, this training also includes a level of background noise.

Back Gap Detection Training—Temporal Skills Module

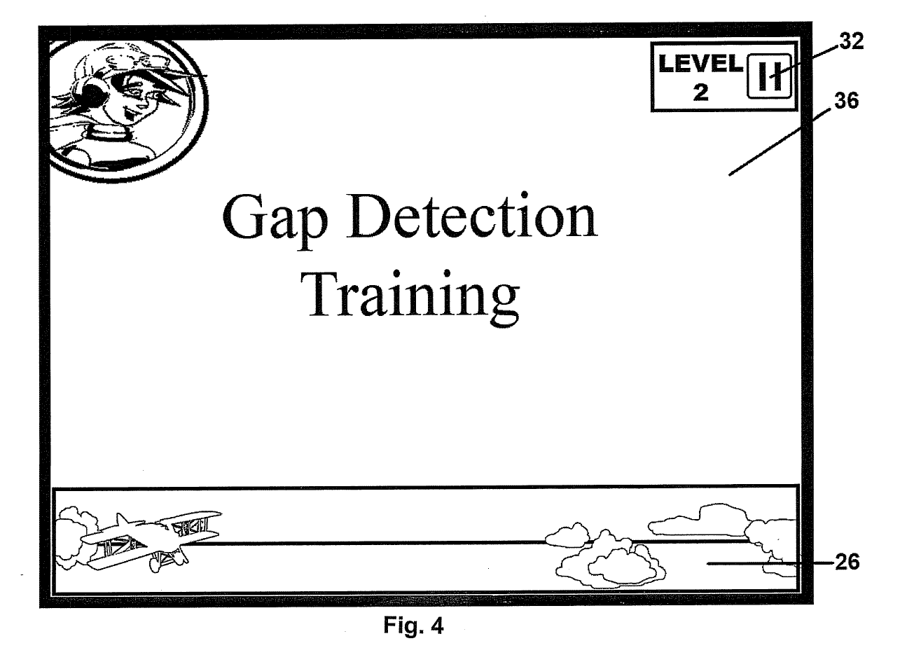

In FIG. 4, shown is a screen shot for the gap detection training. The screen shot is indicated by numeral 36 and similar to FIG. 3, includes a level indicator 32 and a progress bar or metre 26.

A child's listening skills are controlled by the listening part of the brain in the temporal lobe. To help improve the child's ability to listen more effectively, this part of the brain can be “trained”. Gap detection is a very effective way to test and enhance auditory temporal processes.

The goal of the module is to improve auditory processing in the temporal lobe of the child's brain and to assess the child's ability to recognize periods of silence (gaps) between pairs of tones as well as improve the child's attention skills. It has been found that this module is particularly useful for helping children with ADHD.

There are 37 levels with 10 gap detection tests per level. The methodology involves the presentation of one continuous toning interrupted with various random periods of silence. Levels increase in difficulty by decreasing the lapse time of the individual gaps. For each gap recognized in any given test, the child presses the space bar on the keyboard (not shown). This also includes, similar to the other modules, background noise.

Frequency Pattern Training—Temporal Skills Module

In this module, a frequency pattern test is conducted. The purpose of the module is to assess and enhance the child's ability process non-verbal auditory signals as well as to be able to recognize the order and pattern of non-verbal auditory signals as they are being presented in groups of varying frequencies and durations. The further purpose is to enable the listener to distinguish between high and low pitch, which can significantly improve spelling ability and finally help children receive and process each message as it is intended and get more out of verbal presentations. There are 100 levels in the module with 10 frequency pattern tests per level. The protocol involves different sequences of three tone bursts being presented to the left and right ears. Each test contains a combination of three high and low frequency tones to create the patterns. The child hears patterns, such as high-high-low or low-high-low and is asked to duplicate the patterns using the high and low arrow keys.

Rhythm Pattern Training—Temporal Skills Module

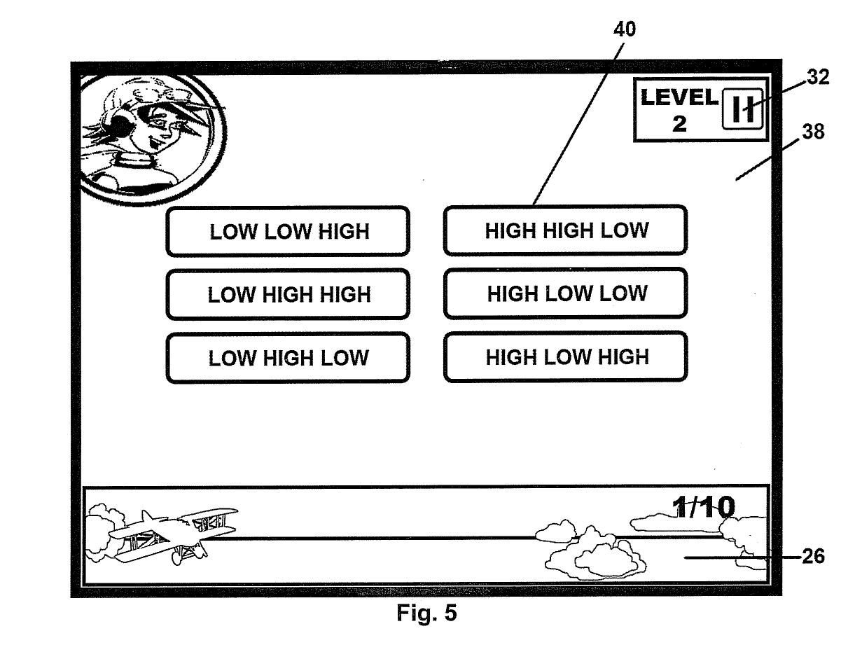

This module incorporates drum patterns tests (rhythm patterns in a temporal pattern/auditory memory enhancement task. The purpose of the module is to enable identification of sound patterns in sequences by using various rhythmic sounds and to improve sound processing and enhance ability to hear spoken messages while building a working memory capacity. This training also improves the child's ability to retain spoken information and improves attention span. FIG. 5 illustrates the screen shot for the frequency matter training, designated by numeral 38. In this module, the screen has frequency patterns 40 that illustrated thereon.

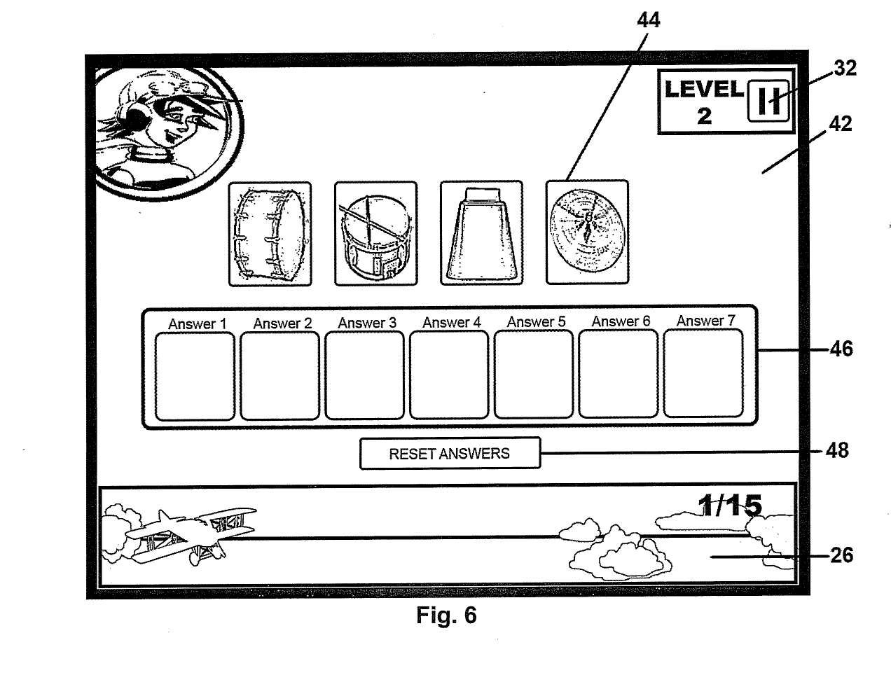

In FIG. 6, shown is the screen shot 42 for the rhythm pattern training. Similar numerals denote similar components from previous figures. In this screen shot, a series of images 44 are presented together with answer blocks, shown in the example is answer blocks 1 through 7 denoted by numeral 46 together with a “reset answers” function 48.

The protocol involves 83 levels with 15 rhythm pattern tests in each level. Sounds include four basic instrumental sounds, examples of which include bass drum, snare drum, cymbals and cow bell. The method involves presenting the child with various tapped rhythms and the child is asked to repeat the pattern of sounds heard. The tapped patterns become increasingly complex as the child advances through the various levels. Additional percussion sounds are added and the number of beats increased to a maximum of six beats per task. Introductory levels contain two, three or four occurrences of two of the bass instruments, whereas intermediate levels contains the same amount of occurrences of three bass instruments and finally advanced levels contain the same amount of occurrences of all four bass instruments.

What is claimed is:

1. A method for diagnosing and treating an auditory processing disorder, comprising:
a diagnosis step and a treatment step, the said diagnosis step including:
presenting, simultaneously at least one different audible sound in a user's left ear and right ear, said sound being different for each said ear;
determining if said user can correctly recall said sound in each ear at that end, whereby incorrect recollection indicates a disorder;
a treatment step including:
repetitively presenting over time, said user with different audio signals to each ear, said signals being presented simultaneously;
providing an interface for the user to attempt to reproduce said audio signals presented; and
monitoring the user's ability to accurately reproduce the correct signals over a period of time.

2. The method as set forth in claim 1, wherein said audible sounds are paired and different within a pair and between pairs.

3. The method as set forth in claim 2, wherein said pairs of audible sounds comprises a dichotic listening analysis.

4. The method as set forth in claim 1, wherein said pairs of audible sounds vary in pitch.

5. The method as set forth in claim 1, wherein said pairs of audible sounds vary in frequency.

6. The method as set forth in claim 1, wherein said pairs of audible sounds comprise numbers.

7. The method as set forth in claim 1, wherein said pairs of audible sounds comprise words.

8. The method as set forth in claim 7, wherein said interface comprises a monitor displaying numbers for selection by said user.

9. The method as set forth in claim 1, wherein said interface comprises a monitor displaying images corresponding to said words for selection by said user.

10. The method as set forth in claim 1, further including providing multiple levels in the presentation of audible sounds to said user.

11. The method as set forth in claim 10, wherein said levels increase in complexity of said audible sound presented.

12. The method as set forth in claim 1, further including providing a scoring meter on said interface for displaying the success in responses from said user.

13. The method as set forth in claim 1, further including providing background noise during presentation of audible sounds.

14. A method for diagnosis and treating auditory processing disorder, comprising: a diagnosis step and a treatment step, said diagnosis step including:
determining which, if any ear is dominant for hearing in a user by presenting, simultaneously at least on different audible sound in a user's left ear and right ear, said sound being different for each said ear; and
determining if said user can correctly recall said sound in each ear, whereby incorrect recollection indicates a disorder indicating a dominant hearing ear;
a treatment step including;
repetitively presenting over time said user with different audio signals to each ear, said signals being presented simultaneously, presentation being effected in ascending levels with increasingly complex sounds;
varying the volume of repeated signals; providing an interface for the user to attempt to reproduce said audio signals presented; monitoring the user's ability to accurately reproduce the correct signals in each level of said levels, whereby ear dominancy in said user is substantially eliminated.

15. The method is set forth in claim 14, wherein said interface comprises a touch sensitive interface.

16. The method is set forth in claim 15, wherein said interface comprises a screen.

17. The method is set forth in claim 15, wherein said interface comprises a wireless pad.

18. The method is set forth in claim 14, further including providing background noise during presentation of audible sounds.

19. The method is set forth in claim 15, further including providing a scoring meter on said interface for displaying the success and responses from said user.
